(19) United States
(12) Patent Application Publication
(10) Pub. No.: US 2015/0119784 A1
(43) Pub. Date: Apr. 30, 2015

(54) Cerumen Cleaner (Earwax Cleaning Device)

(71) Applicant: Terata Kanu, Okc, OK (US)

(72) Inventor: Terata Kanu, Okc, OK (US)

(21) Appl. No.: 14/067,860

(22) Filed: Oct. 30, 2013

(51) Int. Cl. A61F 13/40

(52) U.S. Cl. CPC A61M 35/006
USPC 604/3

(57) ABSTRACT

Most cotton swabs, that currently exist, are not designed for cleaning the external ear canal. The risk of eardrum perforation and/or earwax impaction is too great. This invention is unique in the sense that it is specially designed to clean the external ear canal. It is precisely designed to fit the external ear canal by measuring 2.54 centimeters in length, which is the size of the average adult ear canal. It is also distinctively designed in the sense that is utilizes the antiseptic effects of hydrogen peroxide to dissolve earwax (cerumen), and cleanse the ear of harmful bacteria.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

1. Field of Invention

This invention relates to devices for cleaning cerumen (earwax) from the outer ear canal, and partially as an antiseptic agent for external ear canal, to include tympanic membrane.

2. Description of Prior Act

Q-Tips are probably the most well known product used for cleaning the ear and removing earwax from the external ear canal. This device is made of tightly bound cotton swabs fixed on an elongated stem, usually approximately 3 inches in length. The problem with such a device is that users have a tendency to insert the cotton swab too far into their ear canal damaging the tympanic membrane (ear drum) and sometimes causing a perforation which can have long term sequelae. This is why most cotton swab manufacturers print a warning on the boxes that states: “Do not insert swab into ear canal.”Moreover the traditional cottons swabs actually cause cerumen impaction instead of removing the earwax.

### SUMMARY OF INVENTION

In accordance with the statues of this present invention, it will be a disposable earwax cleaning product, approved for external ear canal cleaning. It is composed of a 2.54 cm partially hollow shaft, the average length of an adult human external ear canal, It is fitted with a tightly wound cotton tip on the distal end, and a plastic or rubber bulb on the proximal end. The bulb contains hydrogen peroxide, and when squeezed, distributes the liquid antiseptic solution down the shaft, into the distal end saturating the cotton swab tip. Once the cotton swab end is saturated, the stem can be inserted into the external ear canal. Because of the device's length, shaft only 1 inch (2.54 cm), the risk of perforating the tympanic membrane is very low. Once the hydrogen peroxide reacts with the cerumen, a mild oxidative reaction will take place, cleaning the ear canal and ear drum (tympanic membrane). The antiseptic effects of the hydrogen peroxide will also act to remove harmful germs and bacteria.

### BRIEF DESCRIPTION OF THE SEVERAL VIEWS OF THE DRAWING

FIG. 1: is a perspective view of the invention.

FIG. 2: is a side elevation view with parts broken away.

FIG. 3: is a side elevation view with parts broken away.

FIG. 4: is a typical cross-sectional view of an ear canal with the invention in use.

### DESCRIPTION LIST

10: is the overall earwax cleaning device invention.

12: is a rubber bulb.

14: is a tube.

16: is a cotton swab tip.

18: is a diaphragm.

18a: is the ruptured diaphragm.

20: is the hydrogen peroxide.

22 is an ear.

24: is the ear canal.

### DETAIL DESCRIPTION OF DRAWING

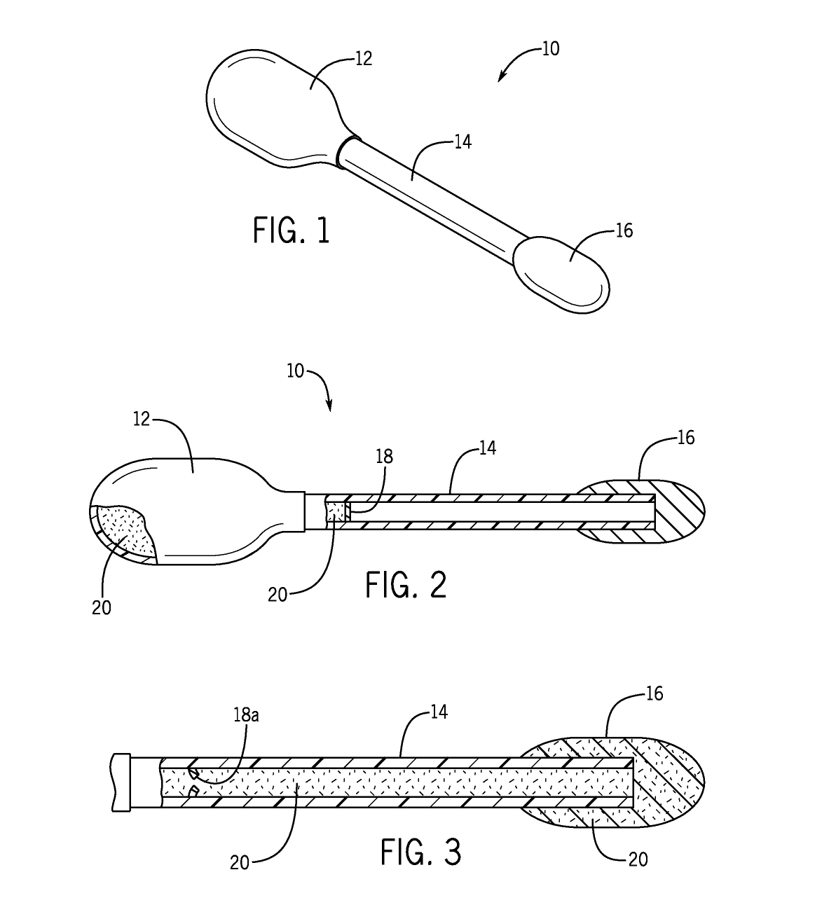

1. FIG. 1 shows the entire invention (part 10) and its individualized parts. Part 12 is the rubber bulb which contains the hydrogen peroxide. Part 14 is the partially hollow tube, approximately 2.54 cm. Part 16 is the cotton swab tip.

2. FIG. 2 further breaks down the invention. Part 20 represents the hydrogen peroxide. Part 18 represents the thin diaphragm or filament barricading the hydrogen peroxide into rubber bulb until it is ready for use. Part 14 showing the partially hollow tube, which will transport the hydrogen peroxide once the rubber bulb (part 12) is squeezed.

3. FIG. 3 illustrates the rubber bulb (part 12) being squeezed, the thin diaphragm (part 18) being ruptured, and hydrogen peroxide traveling down the partially hollow tube. Once released the hydrogen peroxide will saturate the cotton tip end (part 16). And the invention will be ready for use.

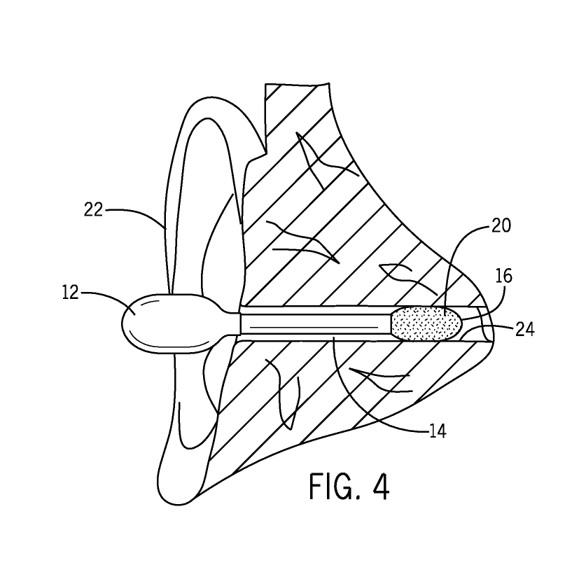

4. FIG. 4 represents the invention being used to clean the external ear canal (part 24). Part 22 represents the external ear. The hydrogen peroxide saturated cotton tip (parts 16 & 20) will cause a mild oxidative reaction with the earwax (cerumen). This will dissolve the cerumen, and clean the external ear.

### DETAILED DESCRIPTION OF THE INVENTION

1. This device is used as an earwax cleaning product. It is comprise of a 2.54 cm partially hollow shaft, fitted with a bulb apparatus at the proximal end. The bulb apparatus contains a small portion of liquid hydrogen peroxide. The distal end of the shaft is fitted with tightly wound cotton tip. When the bulb end is squeezed, the thin filament or diaphragm blocking off the hydrogen peroxide from the shaft is broken. Hydrogen peroxide then proceeds to flow into the partially hollow shaft saturating the distal end containing the cotton tip. Once saturated the device can be inserted into the external ear canal where the hydrogen peroxide acts to dissolve cerumen, and then cleanse and sterilize the external ear canal.

2. This invention has resemblance to a traditional Q-tip, but is greatly unique in the sense that it is specially crafted for the external canal, measuring 2.54 cm in the average adult. This invention is also unique because it utilizes the antiseptic agent hydrogen peroxide to clean and sterilize the external ear canal.

3. This invention will be made with plastic or rubber to craft the bulb end. The shaft or partially hollow tube will be made of plastic or paper material. The distal end will be made of tightly wound cotton to form a tip or swab.

4. Initially, the product will be constructed by a product engineer. Then once a prototype is built, mass production of the product can commence at a manufacturer. This product is intended for personal hygiene uses. Consumers can use this product to clean their ears on a daily basis.

1. This device is used as an earwax cleaning product. It is comprise of a 2.54 cm partially hollow shaft, fitted with a bulb apparatus at the proximal end. The bulb apparatus contains a small portion of liquid hydrogen peroxide. The distal end of the shaft is fitted with tightly wound cotton tip. When the bulb end is squeezed, the thin filament or diaphragm blocking off the hydrogen peroxide from the shaft is broken. Hydrogen peroxide then proceeds to flow into the partially hollow shaft saturating the distal end containing the cotton tip. Once saturated the device can be inserted into the external ear canal where the hydrogen peroxide acts to dissolve cerumen, and then cleanse and sterilize the external ear canal.

2. The device of claim 1 wherein says shaft, is approximately 2.54 cm in length

3. The device of claim 1 wherein says shaft, is made of plastic or paper.

4. The device of claim 1 wherein says bulb, is made of plastic or rubber and filled with hydrogen peroxide.

5. The device of claim 1 wherein says distal end, is made of a cotton swab tip.
